(19) United States
(12) Patent Application Publication
(10) Pub. No.: US 2007/0104859 A1
(43) Pub. Date: May 10, 2007

(54) Coating for environmental protection and indication

(76) Inventors: Michael Featherby, San Diego, CA (US); Carl S. Edwards, San Diego, CA (US)

Correspondence Address: NATH & ASSOCIATES PLLC, 112 South West Street, Alexandria, VA, 22314, US

(21) Appl. No.: 11/431,010

(22) Filed: May 10, 2006

Related U.S. Application Data
(60) Provisional application No. 60/679,536, filed on May 10, 2005.

(51) Int. Cl. A61L 33/00; B05D 3/02; B05D 3/00; C09D 1/00

(52) U.S. Cl. 427/2.1; 427/384; 106/286.5; 106/287.17; 106/287.1; 106/286.2; 252/301.4 R; 252/301.4 F; 106/286.4; 106/623; 106/632; 106/635

(57) ABSTRACT

A composition for coating a surface comprises an adhesive cementitious material such as a geopolymer, a kaolin, or mixtures thereof, and a filler material. Various filler materials are disclosed, with which the coating composition may be used for thermal mapping, oxygen protection to organic dopants, thermal protection, radiation protection, anti-tamper protection, mechanical abrasion protection, or high emission of electromagnetic radiation. With certain fillers, the coating composition remains thermally stable up to 1200° C., and/or remains thermally operable for brief periods up to about 1400° C. When the coating composition includes a wetting agent, and the filler is between 0.01 micrometer and 10 micrometers in size, the coating composition can take the form of an aqueous spray. With certain fillers, the coating composition can cure at a temperature below 100° C. The coating composition can also be a thick film which provides resistance to cracking and peeling during severe thermal exposure.

This application claims the benefit of Provisional U.S. Patent Application 60/679,536 filed May 10, 2005, the contents of which are hereby incorporated by reference in their entirety.

### FIELD OF THE INVENTION

The present inventive subject matter relates to coatings for environmental protection and indication.

### BACKGROUND OF THE INVENTION

Many fields benefit from coatings for environmental protection and indication. As a non-limiting example, aerospace engineers seek advanced coatings to protect vehicles, projectiles, and their components, from temperature, pressure, radiation, abrasion, and tampering. In addition, aerospace engineers often test vehicles and projectiles in hypersonic wind tunnels, where the engineers desire coatings which provide indication of temperature and pressure at the surface of the vehicle or projectile. Such wind tunnel tests often further necessitate the removal of the coating from the surface of the vehicle or projectile after testing. Similar needs are felt in many other fields where environmental protection and indication are useful, including as non-limiting examples mechanical, environmental, automotive, electrical, and chemical engineering; materials science; manufacturing; and innumerable military endeavors.

Organic coatings have been tried for these purposes. Sometimes these coatings are purely organic agents for environmental protection and indication, and sometimes the agent for environmental protection and indication is suspended in an organic matrix material. However, organic coatings for these purposes have weaknesses, most notably associated with low thermal stability of the organic materials. Organic bonds tend to decompose at temperatures above 400° C., and sometimes the organic materials begin to decompose and outgas at even lower temperatures. As a result, useful limits for organic coatings are severely constrained at high temperatures. Further, removal of organic coatings for wind tunnel models requires the use of toxic chemicals, and the coating is often not reusable upon removal. Additionally, organic coatings can interact undesirably with the surface to which they are applied. Organic coatings can be difficult to apply, costly to produce, and can require high temperatures for curing (far above 100° C.). Organic coatings can show poor thermal resolution, failing to accurately indicate thermal changes around certain temperatures or above and below known temperature ranges. Organic coatings poorly handle thermal cycling, or repeated changes in temperature.

### SUMMARY OF THE INVENTION

There is therefore a need for a coating which can withstand high temperatures while maintaining its structure and continuing to perform its function; which is easy to apply and can be removed without toxic chemicals; which can be reused upon removal; which can be applied to a surface without undesirable interactions at the surface; which is easy to apply; which is inexpensive to produce; which can cure at temperatures around or below 100° C.; which show good thermal resolution at both high and low temperatures; and which can handle thermal cycling.

The present inventive subject matter addresses these needs through the manufacture and use of coating materials which employ inorganic matrix materials which provide the necessary characteristics described above. Non-limiting examples of such inorganic matrix materials include geopolymers and kaolins.

One embodiment of the present inventive subject matter provides a composition for coating a surface. The composition comprises an adhesive cementitious material such as a geopolymer, a kaolin, or mixtures thereof, and up to 70% by final volume of a filler material.

In another embodiment, the filler material is calcium silicate, titania, zirconia, alumina, silicon carbide, luminophores, carbon or mixtures thereof. This coating composition may be used for thermal mapping by applying the coating composition to a surface to form a coated article, curing the coated article, allowing the temperature of the surface to change, and observing changes in the coating composition as the surface changes temperature. This coating composition can provide oxygen protection to organic dopants (such as the non-limiting examples of luminophores and phosphores) by applying the coating composition with the organic dopant to form a thermally stable structure, and curing the coated article, thereby providing environmental and thermal stability and oxidization protection to sensitive organic materials during environmental exposure.

In yet another embodiment, the filler material is titanium, zirconium, aluminum, silicon, iron, copper, nickel, cobalt, silicon, hafnium, oxides thereof, carbides thereof, nitrides thereof or mixtures thereof, and the coating composition remains thermally operable for brief periods at temperatures up to about 1400° C.

In still another embodiment, the filler material is silica, alumina, titania, zirconia, glass microspheres, carbon and mixtures thereof. This coating composition may be used to provide thermal protection by applying the coating composition to an article to form a coated article, and curing the coated article, thereby providing thermal protection for the coated article.

In still another embodiment, the filler material is tungsten, titanium, gadolinium, hafnium, lead, boron, oxides thereof, carbides thereof, nitrides thereof, or mixtures thereof. This coating composition may be used to provide radiation protection to military or medical components by applying the coating composition to the military or medical components to form a coated article, and curing the coated article, thereby providing radiation protection for the coated article.

In still another embodiment, the filler material is silicon, aluminum, titanium, zirconium, oxides thereof, carbides thereof, nitrides thereof, carbon nanotubes or mixtures thereof. This coating composition may be used to provide anti-tamper protection by applying the coating composition to an article to form a coated article, and curing the coated article, thereby providing anti-tamper protection for the coated article.

In still another embodiment, the filler material is calcium silicate, titania, zirconia, alumina, silicon carbide, luminophores, silica, silicates, ceramics, iron, copper, nickel, cobalt, silicon, aluminum, titanium, oxides thereof, carbides thereof, nitrides thereof, or mixtures thereof. This coating composition may be used to provide mechanical abrasion protection by applying the coating composition to an article to form a coated article, and curing the coated article, thereby providing mechanical abrasion protection for the coated article.

In still another embodiment, the filler material has high emissivity, and may be calcium silicate, silicon carbide, zirconia, alumina, titania, or mixtures thereof. This coating composition may be used to provide high emission of electromagnetic radiation by applying the coating composition to an article to form a coated article, and curing the coated article, thereby providing high emission of electromagnetic radiation for the coated article.

In still another embodiment, the composition is thermally stable up to about 1400° C.

In still another embodiment, the coating composition includes no more than 0.5% of a wetting agent by weight, and the filler is between 0.01 micrometer and 10 micrometers in size, such that the composition is in the form of an aqueous spray. In still another embodiment, the composition may be cured by depositing the composition on a surface at a thickness of no more than 0.010 inches, and allowing the composition to cure at ambient temperatures, or at a temperature of up to about 100° C.

In still another embodiment, the coating composition is a thick film which provides resistance to cracking and peeling during severe thermal exposure.

These and other aspects and features of the invention will be better understood by those of skill in the art with reference to the following figures and description wherein like numbers represent like objects throughout the several views.

### BRIEF DESCRIPTION OF THE FIGURES

FIG. 1 is a perspective view of an embodiment of the present inventive subject matter applied to a substrate.

### DETAILED DESCRIPTION OF THE INVENTION

The present inventive subject matter is directed to a composition for coating a surface. The composition comprises an adhesive cementitious material such as a geopolymer, a kaolin, or mixtures thereof, and up to 70% by final volume of a filler material.

Geopolymers are inorganic polymeric ceramics with a polysiliate microstructure of linked SiO₄ and AlO₄ tetrahedral material. Geopolymers can also contain magnesium-silicate. Geopolymers are typically formed from the geosynthesis of polymeric alumino-silicates and alkali-silicates under highly alkaline conditions at ambient temperatures. Geopolymers share their form with naturally occurring silicates such as clay, micas, mullite, andalusite, spinel, and flyash. Geopolymers are the amorphous equivalent of zeolites.

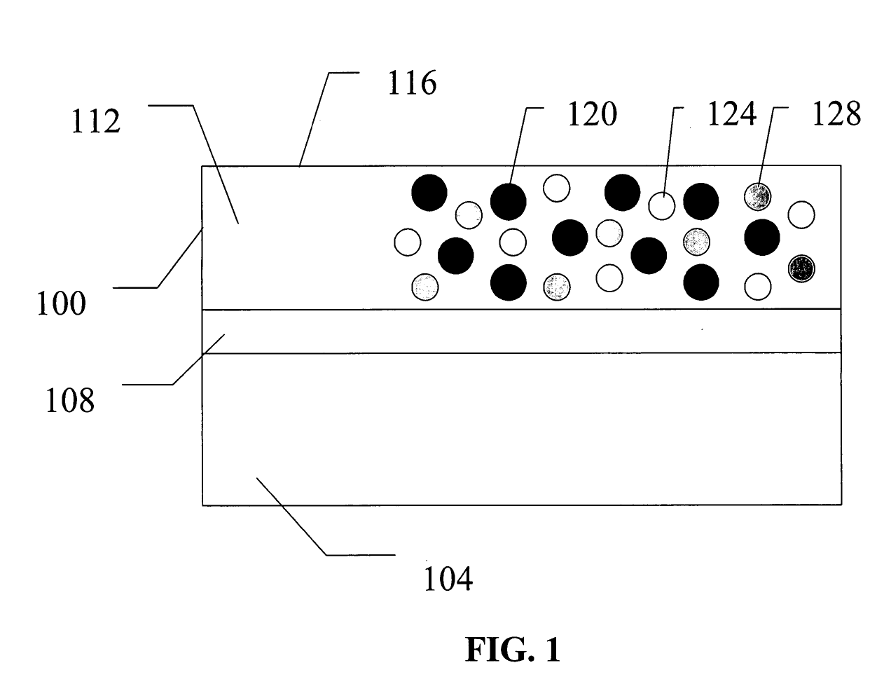

As shown in FIG. 1, a coating 100 which includes a cementitious material 112 is coated onto a substrate 104. The cementitious material 112 is chosen for its physical and chemical characteristics, which can be controlled by techniques such as the non-limiting examples of varying the ratio of AlO₄ tetrahedral units to SiO₄ tetrahedral units, or forming the cementitious material 112 from one or more naturally occurring silicates.

The coating 100 can be applied through many different techniques. The choice of techniques depends on the structure of the cementitious material 112, the fillers 120, 124, 128 and any other agents with which these are combined in the coating before deposition. As a non-limiting example, the cementitious material 112 can be incorporated with a wetting agent, and if all fillers 120, 124, 128 are maintained at a size of no more than about 10 micrometers the combined coating can be spray coated onto the substrate 104. Other non-limiting examples of deposition techniques include coating by brush, flame spray coating, substrate immersion, stenciling, silk screening, doctor blading, and various other deposition techniques known in the art. The following table sets forth non-limiting examples of average thicknesses at which such methods deposit a coating.

| Method | Thickness (inches) |
|---|---|
| Liquid Spray | about 0.001-0.003 |
| Stencil Print | about 0.001-0.005 |
| Doctor Blade | about 0.001-0.010 |
| Pad Print | about 0.001-0.005 |

The inventive compositions can be applied such that the coatings are either considered thick or thin coatings. As used herein, “thick” refers to coatings having a thickness of about 0.005 inches to about 0.010 inches. Likewise, as used herein, “thin” refers to coatings having a thickness of about 0.001 inches to about 0.045 inches.

The substrate 104 substrate 104 is any material onto which the coating 100 is to be cured. Non-limiting examples of substrates 104 include aluminum, copper, cobalt, nickel, iron, steel, bondo, glass, plastics, ceramics, or any other material to which a cementitious material 112 will adhere. Once deposited, the coating must be cured. Many geopolymers 112 provide for curing at ambient temperatures, or at temperatures below 100° C. The curing step is generally done at an elevated temperature for a duration sufficient to cure the coating. For example, the curing step may be done at a temperature of about 25° C. (±15° C.) for about 60 to about 120 minutes to form the final product. If the curing step is done at a higher temperature, then a shorter curing time is required. For instance, if the curing step is done at a temperature of about 75° C. (±25° C.), then the coated article is cured for about 15 to about 45 minutes in order to form the final product.

The coating 100 and substrate 104 meet at an interface region 108. Proper bonding at the interface region 108 and mechanical stability of the coating 100 assure that the coating 100 will not delaminate, which would result in spallation, peeling, or flaking of the coating 100. Unlike with many organic coatings, bonding at the interface region 108 can be made so that the coating 100 is removable without the use of toxic chemicals. Moreover, the cementitious material 112 and fillers 120, 124, 128 can be chosen to be mechanically reusable after removal.

FIG. 1 depicts a non-limiting example of an embodiment of the present inventive subject matter. In FIG. 1, three different types of fillers 120, 124, 128 are shown. However, one of skill in the art will easily recognize that any number of fillers can be included in the inventive compositions. As indicated above, the number of types of fillers present will be dictated by the mechanical and other properties desired for particular use of the coating. The cementitious material 112 serves as an inorganic matrix material for suspending one or more fillers 120, 124, 128. For example, emitter fillers are shown by reference numeral 124. Emitter fillers 124 can serve to emit signals in the presence of changes at the substrate 104. Such signals are typically emitted at the top surface 116. Non-limiting examples of such signals include electromagnetic radiation and chemical signals. Non-limiting examples of such changes at the substrate include changes in heat, pressure, torsion, and other forces, chemical changes, changes in absorption rates of various gases or liquids, and numerous other changes to a material, known in the art, which can be observed in this fashion. Thus, the emitter fillers 124 emit signals in response to those changes. Non-limiting examples of such fillers include calcium silicate, titania, zirconia, alumina, silicon carbide, luminophores, carbon and mixtures thereof. As used herein, “carbon” refers to elemental carbon in various forms. Non-limiting examples of elemental carbon forms included in the coverage of the term “carbon” include carbon black, glassy carbon, carbon fiber, and mixtures thereof.

This coating composition can provide oxygen protection to organic dopants (such as the non-limiting examples of luminophores and phosphores) by applying the coating composition with the organic dopant on an article, and curing the coated article, thereby providing environmental and thermal stability and oxidization protection to sensitive organic materials during environmental exposure.

More than one emitter filler 124 may be used in one coating 100, and proper combinations of emitter fillers can provide enhanced signal emission, or the ability to simultaneously record signals for disparate changes at the substrate 104.

The choice of cementitious material 112 and fillers 120, 124, 128 can determine the temperatures at which the coating 100 is structurally stable, and at which the emitter fillers 124 remain thermally operable. A thermally operable emitter filler 124 is one which continues to emit radiation corresponding to temperature and/or temperature changes. Unlike organic coatings, which often thermally decompose at temperatures above 400° C., geopolymers 112 can often withstand severe thermal exposure (“severe” thermal exposure being exposure to temperature above 900° C.), and can remain structurally stable at temperatures up to 1200° C. or even 1400° C. for brief periods of time. The following table sets forth non-limiting examples of the average time periods over which coatings retain their structural integrity at a given temperature.

| Temperature (° C.) | Duration |
|---|---|
| 700 | about 8 hours |
| 1000 | about 20 minutes |
| 1400 | about less than 5 minutes |

Moreover, by remaining structurally stable, the cementitious material 112 can continue to provide support and protection for emitter fillers 124, allowing them to continue to emit signals reflective of changes (thus, a thermally operable emitter filler emits signal reflective of temperature changes even at these high temperatures). Non-limiting examples of emitter fillers 124 that allow the coatings to remain thermally operable at temperatures up to 1400° C. include titanium, zirconium, aluminum, silicon, iron, copper, nickel, cobalt, silicon, hafnium, oxides thereof, carbides thereof, nitrides thereof and mixtures thereof.

A further embodiment of the present inventive subject matter presents a coating 100 for thermal mapping. The coating 100 contains an emitter filler 124 which can emit electromagnetic radiation (in the form of visible light, infrared energy, ultraviolet energy, or other radiation) as an indication of the temperature, or changes in temperature, of the substrate 104 to which is it applied. Alternately, the coating 100 can contain an emitter filler 124 which changes color (by changing its absorptive or reflective properties) as a reflection of the temperature, or changes in temperature, of the substrate 104 to which it is applied. Non-limiting examples of an appropriate emitter filler 124 include calcium silicate, titania, zirconia, alumina, silicon carbide, luminophores, carbon and mixtures thereof. Proper proportions of emitter fillers can produce a coating 100 with high emissivity. This coating composition 100 may be used for thermal mapping by applying the coating composition 100 to the substrate 104 to form a coated article, curing the coated article, allowing the temperature of the surface to change, and observing changes in the coating composition as the surface changes temperature. Non-limiting examples of surfaces with substrates 104 to which such a coating 100 may be usefully applied include the outside surfaces of aircrafts, missiles, and space vehicles; objects to be sent into aerospace or space environments such as those subjected to hypersonic speeds in the lower atmosphere; objects to be places in hypersonic wind tunnel tests; power stations; engine rooms; and articles for use in water based applications.

In the present inventive embodiments in which the inventive compositions are used in thermal mapping and emitter applications, the emitter wavelengths may be in the optical or short-wave infrared (IR) ranges. For example, if the emitter wavelength is in the optical range, the emitter wavelength will be about 380 to about 780 nanometers. Likewise, if the emitter wavelength is in the short-wave IR range, the emitter will have a wavelength of about 700 nanometers to about 14 microns.

Another example of a possible filler usable in the present inventive subject matter are protection fillers 128. Protection fillers 128 are used to create a protective coating 100, which can protect the substrate 104 from adverse conditions encountered at the top surface 116. Non-limiting examples of adverse conditions include environmental exposure, heat, cold, changes in temperature, radiation, tampering, and abrasion. Non-limiting examples of such fillers which provide suitable protection include alumina, aluminum, boron, calcium silicate, ceramics, cobalt, copper, gadolinium, glass micro spheres, hafnium, iron, lead, nickel, silica, silicates, silicon carbide, silicon, syntactic glass, titania, titanium, tungsten, zirconia, zirconium, and mixtures thereof. More than one protection filler 128 may be used in one coating 100, and proper combinations of protection fillers can provide enhanced protection, or simultaneous protection from more than one adverse condition at the substrate 104.

Additional structure fillers 120, such as the wetting agent set forth as a non-limiting example above, can also be included to add physical attributes or other desirable thermal or mechanical properties to the coating. Non-limiting examples of such fillers include silicon carbide, calcium silicate, zirconia, titania, alumina, syntactic glass, and mixtures thereof. More than one structure filler 120 may be used in one coating 100, and proper combinations of structure fillers can provide enhanced structural capacities to the coating 100, or allow the coating 100 to change structure properties under different conditions. Non-limiting examples of such conditions include temperature changes, pressure changes, and exposure to electromagnetic radiation.

Another embodiment of the present inventive subject matter presents thermal protection to structures by applying the coating composition 100 to the surface or exterior of a structure as a substrate 104 to form a coated article, and then curing the coated article, thereby providing an article with a coating providing enhanced thermal protection. Non-limiting examples of an appropriate protection filler 128 for thermal protection include silica, alumina, titania, zirconia, glass microspheres, carbon and mixtures thereof. Non-limiting examples of appropriate structures for which thermal protection might be sought include high temperature surfaces, automotive under-hood applications, power plant engine rooms, commercial aerospace applications, military aerospace applications, printed circuit boards, and other applications with severe environments.

Another embodiment of the present inventive subject matter presents radiation protection to structures by applying the coating composition 100 to the surface or exterior of a structure as a substrate 104 to form a coated article, and then curing the coated article, thereby providing an article with a coating providing enhanced radiation protection. Non-limiting examples of an appropriate protection filler 128 for radiation protection include tungsten, titanium, gadolinium, hafnium, lead, boron, oxides thereof, carbides thereof, nitrides thereof, and mixtures thereof. Non-limiting examples of appropriate structures for which radiation protection might be sought include medical environments, medical equipment, nuclear power plants, equipment in other nuclear environments, and printed circuit boards.

Another embodiment of the present inventive subject matter presents anti-tamper protection to structures by applying the coating composition 100 to the surface. or exterior of a structure as a substrate 104 to form a coated article, and then curing the coated article, thereby providing an article with a coating providing enhanced anti-tamper protection. Non-limiting examples of an appropriate protection filler 128 for anti-tamper protection include silicon, aluminum, titanium, zirconium, oxides thereof, carbides thereof, nitrides thereof, carbon nanotubes and mixtures thereof. Non-limiting examples of appropriate structures for which anti-tamper protection might be sought include computer chips, printed circuit boards, or other structures for which reverse engineering may be pursued if not adequately protected. The application of the coating can prevent the contents of the structure from being x-rayed, and/or from being dismantled by chemical or mechanical means. As a non-limiting example, circuitry which can typically be reverse engineered by chemical etching can be made tamper proof by the application of an inorganic coating which is removed by the use of chemicals known to damage the circuitry underneath.

Another embodiment of the present inventive subject matter presents mechanical abrasion protection to structures by applying the coating composition 100 to the surface or exterior of a structure as a substrate 104 to form a coated article, and then curing the coated article, thereby providing an article with a coating providing enhanced mechanical abrasion protection. Non-limiting examples of an appropriate protection filler 128 for mechanical abrasion protection include calcium silicate, titania, zirconia, alumina, silicon carbide, luminophores, silica, silicates, ceramics, iron, copper, nickel, cobalt, silicon, aluminum, titanium, oxides thereof, carbides thereof, nitrides thereof, and mixtures thereof. Non-limiting examples of appropriate structures for which mechanical abrasion protection might be sought include automotive under-hood applications, power plant engine rooms, printed circuit boards, commercial aerospace applications, and military aerospace applications.

As previously indicated, the present inventive subject matter includes an adhesive cementitious material selected from the group consisting of geopolymer, kaolin, and mixtures thereof. In those embodiments in which geopolymer is used as the cementitious material, the geopolymer is preferably present in the form of a combination with water. The combination of geopolymer with water is preferably within the range of a molar ratio of about 7:1 to about 15:1 of H₂/R₂O, wherein R is sodium or potassium. In an alternative embodiment, a geopolymer solution is prepared in which the geopolymer is present with a hardener in a weight ratio of about 40:60 to about 60:40. Various components of the geopolymer solution include R₂O as defined above, Al₂O₃, and SiO₂.

The following example is illustrative of an embodiment of the present inventive subject matter, and sets forth a method for preparing, applying and curing the coating. As other methods can be discerned from the above disclosure by one skilled in the art, this example is not to be construed as limiting the inventive subject matter therein.

### EXAMPLE 1

Initially, one should weigh out the raw materials to be used in the coating. One may use the following, non-limiting examples to set the ratio of Silicate liquid, Silicate powder or fly ash, filler(s), and wetting agent(s).

| Item | Quantity wt. Percent range |
|---|---|
| Sodium or Potassium Silicate liquid | 20-55% |
| Alumino-Silicate powder or fly ash | 10-25% |
| Filler(s) | 20-70% |
| Wetting Agent(s) | 0-0.5% |

Then, using a suitable mixing vessel, one should place the weighed ingredients in a vessel and mix thoroughly, either by hand or machine, at room temperature. The mixing step is finished when all ingredients are wetted out and the batch is not lumpy.

Next, one may apply the coating to a substrate. The following non-limiting examples of application techniques may be used to provide coating of a corresponding thickness.

| Method | Thickness (inches) |
|---|---|
| Liquid Spray | about 0.001-0.003 |
| Stencil Print | about 0.001-0.005 |
| Doctor Blade | about 0.001-0.010 |
| Pad Print | about 0.001-0.005 |

After application, the coating may be cured. As a non-limiting example, one may set an oven at 90° C.+/−10° C., set the coated substrate in the oven, and cure for 45+/−15 minutes. Many other curing techniques are known to those skilled in the art.

After curing, the item may be removed from the oven or other curing apparatus (if so used) and inspected for coating uniformity. At this point, the coating is ready for use.

The following example is illustrative of an embodiment of the present inventive subject matter as tested for its abilities in thermal mapping, and is not to be construed as limiting the inventive subject matter thereto.

### EXAMPLE 2

A multi-channel recorder was used to log thermal results and a high intensity quartz heat lamp was used to supply heat. Candidate materials were evaluated thermally. All samples were prepared on a 6″×3″×0.5″ steel coupon. Each coupon had half of one surface coated with the test material and the other side coated with a control coating of organic binder and a black pigment (muffler paint). A total of 6 thermocouples were mounted with the beads just behind the test surface, from which accurate temperature measurements could be taken.

A geopolymer was evaluated with a SiC filler. The geopolymer was formed, applied, and cured according to the methods given above. The geopolymer created an organic free, thermally resistant chemical bond with the coupon. Optimized formulations were made that demonstrated improved emissivity. Using a high intensity quartz lamp, a 480 second exposure was made. The run incorporated two minutes (120 seconds) with the lamp on, followed by 10 seconds with the lamp off. At the end of each two-minute period, an IR shot was taken of the sample with the lamp on and off. There were four two-minute cycles, with each successive cycle increasing in temperature. Maximum temperature reached was 206.6° C.

The test coupon was set at a 45-degree angle to the lamp, with the leading edge at a distance of 3 inches from the lamp. The images showed an asymmetrical heating pattern of the candidate material versus the control. The control got hotter than the candidate material as the high emissivity geopolymer has higher emittance than the control. That said, there was thermal agreement with an embedded thermocouple (not visible) to within 2° C., after adjusting for coating emissivity. Camera resolution was 0.05° C. and the temperature gradient could be measured by intensity.

The following example is illustrative of the selection of the present inventive subject matter for its characteristics, and is not to be construed as limiting the inventive subject matter thereto.

### EXAMPLE 3

Several material candidates were selected to serve as candidate coating materials for testing the geopolymer based coating, based on known metal emissivity. A sample table of metal emissivity is set forth below.

| | Emissivity @ | Emissivity @ |
|---|---|---|
| Material | Temperature | Temperature |
| Aluminum metal | 0.028 @ 100 C. | 0.06 @ 500 C. |
| Aluminum oxidized | 0.11 @ 200 C. | 0.19 @ 600 C. |
| Copper metal | 0.02 @ 100 C. | 0.15 @ 1083 C. |
| Copper oxidized | 0.6 @ 200 C. | 0.6 @ 1000 C. |
| Cobalt | 0.13 @ 500 C. | 0.23 @ 1000 C. |
| Cobalt Oxidized | 0.06 @ 1000 C. | 0.12 @ 500 C. |
| Nickel | 0.06 @ 100 C. | 0.12 @ 500 C. |
| Nickel Oxidized | 0.37 @ 200 C. | 0.85 @ 500 C. |
| Iron | 0.05 @ 100 C. | |
| Iron Oxidized | 0.74 @ 100 C. | 0.84 @ 500 C. |

A list of candidate coating materials was developed and several material candidates were selected for evaluation for their thermal emissivity. The coatings were direct deposited on a steel test coupon to show ease of application as a thin film. The advantage of this technique was determined to be relative ease of use and the mechanical stability of a thin film material over thicker structures, which can spall, crack and peel. For output measurement, visible and infrared systems were identified for measuring temperature. The coating demonstrated a continuous signal over the test coupon when heated in the manner set forth in Example 1. The selected candidates are listed in the following table.

| Candidate | Sample (° C.) | Control (° C.) | Delta (° C.) |
|---|---|---|---|
| Fe2O3/BMI/Steel | 180.774 | 187.307 | 6.533 |
| Fe2O3/BMI/Kapton/Steel | 157.578 | 161.333 | 3.755 |
| ZrO2/Fe2O3/Steel | 156.4 | 169.56 | 13.16 |
| Wessex M1 | 187.779 | 190.405 | 2.626 |
| Wessex M3 | 165.993 | 177.355 | 11.362 |
| Geopolymer | 164.474 | 171.778 | 7.304 |
| Fe2O3 bare/Steel | 189.058 | 190.528 | 1.47 |
| SiC/Bare/Steel | 157.152 | 167.432 | 10.28 |
| SiC/BMI/Steel (8) | 189.155 | 202.621 | 13.466 |
| SiC/BMI/Steel (6) | 163.388 | 167.12 | 3.732 |
| SiC/BMI/Kapton/Steel | 194.075 | 185.75 | 8.325 |
| SiC/Geopolymer/Steel | 188.818 | 196.13 | 7.312 |

Black muffler paint (organic binder and black pigment) was used as an organic control. The surface emissivity was estimated to be 0.9, based on the emissivity of acrylic. A 2.54 micrometer thick sample of the control was coated onto a 10.16 micrometer thick steel coupon. The table below sets forth properties of the control and the steel.

| Property | Acrylic | Stainless Steel |
|---|---|---|
| Thermal Conductivity (W/m/K) | 0.25104 | 22.928 |
| Density (kg/m\hat{ }2) | 1200 | 7600 |
| Specific Heat Capacity | 1423 | 460.2 |
| Surface Emissivity | 0.9 | 0.05 typical |

Various organic-based and inorganic-based coatings were compared with this control sample. A table benchmarking comparative thermal results appears below. The table shows the relative performance using IR versus thermocouple readings and gave a gross discriminator for the emitter material. The following table sets forth the thermal test results.

| Candidate | Sample (° C.) | Control (° C.) | Delta (° C.) |
|---|---|---|---|
| Geopolymer (Pure) | 164.474 | 171.778 | 7.304 |
| SiC/Geopolymer/Steel | 188.818 | 196.13 | 7.312 |
| Fe2O3/BMI/Kapton/Steel | 157.578 | 161.333 | 3.755 |
| Fe2O3/BMI/Steel | 180.774 | 187.307 | 6.533 |
| SiC/Bare/Steel | 157.152 | 167.432 | 10.28 |
| ZrO2/Fe2O3/Steel | 156.4 | 169.56 | 13.16 |

Based on this data, promising results were obtained with coatings of Geopolymer and SiC. The Geopolymer coating, unlike organic coatings, employs a chemical reaction and can be built and cured below 100° C. and it forms an oxide bond to the coupon. However, most of the coatings highlighted consist of an organic component, such as polyamide film and bismalemide, an epoxy. As already mentioned, inorganic coatings provide advantages over organic coatings in terms of thermal stability and ease of removal. The large delta between the sample and control (muffler paint) indicates an improvement due to better emissivity. Geopolymer can be filled with many kinds of materials, making it an attractive technology for this and other uses. The last two emitter combinations; SiC and ZrO₂/Fe2O₃ were studies without geopolymer coating for comparison sake. Although SiC was used as a filler, other filler materials have desirable properties, as set forth in the following table.

| | | CTE | | | |
|---|---|---|---|---|---|
| | | (coefficient | Melting | | Thermal |
| | Emis- | of thermal | Point | | Conductivity |
| Filler | sivity | expansion) | (° C.) | Density | (W/mK) |
| Zirconia | 0.9 | 12 | 2681 | 5.7 | 22 |
| Titania | 0.9 | 7.14 | 1855 | 4.23 | 10 |
| Alumina | 0.94 | 8 | 2050 | 4 | 40 |
| Syntactic Glass | 0.9 | 1.6 | >1000 | 0.29 | 0.2 |
| Luminophores | N/A | 10 | >600 | 2.9 | 0.2 |
| Geopolymer | 0.92 | 5 | >1200 | 2.0 | 0.06 |
| SiC | 4.3 | 0.96 | 2700 | 3.1 | 50 |

Thus, the geopolymer approach was chosen and optimized for thermal stability by the elimination of organic materials in the adhesive. The large delta between the sample and control indicated an improvement over the control coating due to improved emissivity. The new coatings have no organic component and are thermally stable at temperatures well above 400° C. (to at least 750° C. with the ZrO₂/Fe2O₃/Steel sample), the point where organic materials begin to break down. The geopolymer can be applied using a thin film approach. The material can be easily applied and removed, and can be formulated with filler material designed for improved emissivity, or other desirable thermal and mechanical properties.

The inventive subject matter being thus described, it will be obvious that the same may be varied in many ways. Such variations are not to be regarded as a departure from the spirit and scope of the inventive subject matter, and all such modifications are intended to be included within the scope of the following claims.

What is claimed is:

1. A composition for coating a surface, the composition comprising:
an adhesive cementitious material selected from the group consisting of geopolymer, kaolin, and mixtures thereof; and
up to 70% by final volume of a filler material.

2. The coating composition of claim 1, wherein said filler material is selected from the group consisting of calcium silicate, titania, zirconia, alumina, silicon carbide, luminophores, carbon and mixtures thereof.

3. A method of thermal mapping, the method comprising:
applying the coating composition of claim 2 to a surface to form a coated article,
curing said coated article,
allowing the temperature of said surface to change, and
observing changes in the coating composition as said surface changes temperature.

4. The method of claim 3, wherein said coating composition further comprises at least one organic dopant, and wherein said adhesive cementitious material is selected to provide environmental stability, thermal stability, or oxidization prevention to said organic dopants.

5. The coating composition of claim 1,
wherein said filler material is selected from the group consisting of titanium, zirconium, aluminum, silicon, iron, copper, nickel, cobalt, silicon, hafnium, oxides thereof, carbides thereof, nitrides thereof and mixtures thereof, and
wherein said coating composition remains thermally operable for brief periods at temperatures up to about 1400° C.

6. The coating composition of claim 1, wherein said filler material is selected from the group consisting of silica, alumina, titania, zirconia, glass micro spheres, carbon and mixtures thereof.

7. A method of providing thermal protection, the method comprising:
applying the coating composition of claim 6 to an article to form a coated article, and
curing said coated article,
thereby providing thermal protection for said coated article.

8. The coating composition of claim 1, wherein said filler material is selected from the group consisting of tungsten, titanium, gadolinium, hafnium, lead, boron, oxides thereof, carbides thereof, nitrides thereof, and mixtures thereof.

9. A method of providing radiation protection to military or medical components, the method comprising:
applying the coating composition of claim 8 to said military or medical components to form a coated article, and
curing said coated article,
thereby providing radiation protection for said coated article.

10. The coating composition of claim 1, wherein said filler material is selected from the group consisting of silicon, aluminum, titanium, zirconium, oxides thereof, carbides thereof, nitrides thereof, carbon nanotubes and mixtures thereof.

11. A method of providing anti-tamper protection, the method comprising:
applying the coating composition of claim 10 to an article to form a coated article, and
curing said coated article,
thereby providing anti-tamper protection for said coated article.

12. The coating composition of claim 1, wherein said filler material is selected from the group consisting of calcium silicate, titania, zirconia, alumina, silicon carbide, luminophores, silica, silicates, ceramics, iron, copper, nickel, cobalt, silicon, aluminum, titanium, oxides thereof, carbides thereof, nitrides thereof, and mixtures thereof.

13. A method of providing mechanical abrasion protection, the method comprising:
applying the coating composition of claim 12 to an article to form a coated article, and
curing said coated article,
thereby providing mechanical abrasion protection for said coated article.

14. The coating composition of claim 1, wherein said filler material has high emissivity.

15. The coating composition of claim 14, wherein said filler material is selected from the group consisting of calcium silicate, silicon carbide, zirconia, alumina, titania, and mixtures thereof.

16. A method of providing high emission of electromagnetic radiation, the method comprising:
applying the coating composition of claim 15 to an article to form a coated article, and
curing said coated article,
thereby providing high emission of electromagnetic radiation for said coated article.

17. The coating composition of claim 1, wherein the composition is thermally stable up to 1200° C.

18. The coating composition of claim 1, said composition further comprising:
no more than 0.5% of a wetting agent by weight, and wherein
said filler is between 0.01 micrometer and 10 micrometers in size, such that
the composition is in the form of an aqueous spray.

19. A method of curing the coating composition of claim 1, said method comprising:
depositing the composition on a surface at a thickness of no more than 0.010 inches, and
allowing the composition to cure at ambient temperatures, or at a temperature of no more than 100° C.

20. The coating composition of claim 1, wherein said coating composition is a thick film which provides resistance to cracking and peeling during severe thermal exposure.

21. The coating composition of claim 2, wherein said coating composition is a thick film which provides resistance to cracking and peeling during severe thermal exposure.

22. The coating composition of claim 3, wherein said coating composition is a thick film which provides resistance to cracking and peeling during severe thermal exposure.

23. The coating composition of claim 5, wherein said coating composition is a thick film which provides resistance to cracking and peeling during severe thermal exposure.

24. The coating composition of claim 6, wherein said coating composition is a thick film which provides resistance to cracking and peeling during severe thermal exposure.
